(19) United States
(12) Patent Application Publication
(10) Pub. No.: US 2007/0050013 A1
(43) Pub. Date: Mar. 1, 2007

(54) Venous valve prosthesis and method of fabrication

(75) Inventor: Jeffrey M. Gross, Memphis, TN (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Jeffrey M. Gross

(21) Appl. No.: 11/351,767

(22) Filed: Feb. 10, 2006

Related U.S. Application Data
(60) Provisional application No. 60/713,458, filed on Sep. 1, 2005.

(51) Int. Cl. A61F 2/06

(52) U.S. Cl. 623/1.24; 623/1.31; 600/36; 623/916

(57) ABSTRACT

The invention provides venous valve prostheses design and method of fabrication useful for replacement of venous valves in the treatment of patients. The venous valve prostheses of the invention comprise at least one integrally formed valve with a proximal converging nozzle and/or a distal diverging nozzle to maintain a proper blood flow rate through the valve.

This application is related to and claims priority to U.S. provisional application Ser. No. 60/713,458, filed Sep. 1, 2005, the disclosure of which is incorporated by reference herein.

### FIELD OF THE INVENTION

The invention relates to venous valve prostheses that comprise at least one integrally formed valve with a proximal converging nozzle and/or a distal diverging nozzle to maintain a proper blood flow rate through the valve. The venous valve prostheses of the invention are useful for replacing venous valves in patients in need thereof. The invention particularly relates to methods of treating patients having venous circulation problems, such as chronic venous insufficiency, comprising implanting a venous valve prosthesis of the invention in said patient. The invention further relates to methods for making a venous valve prosthesis of the invention, as well as methods for sizing a venous valve prosthesis of the invention.

### BACKGROUND OF THE INVENTION

Patients with Chronic Venous Insufficiency (CVI) have deep and superficial venous valves of their lower extremities (distal to their pelvis) that have failed due to congenital valvular abnormalities and/or pathophysiologic disease of their vasculature. As a result, these patients suffer from varicose veins, swelling and pain of the lower extremities, edema, hyper pigmentation, lipodermatosclerosis, and deep vein thrombosis (DVT). They are at increased risk for development of soft tissue necrosis, ulcerations, pulmonary embolism, stroke, heart attack, and amputations. CVI is often misdiagnosed and represents an annual cost to the US health care system between $750 Million and $1 Billion dollars (Weingarten, 2001, Clinical Practice 32:949-954), with most of this cost due to the treatment and associated care of chronic ulcerations.

The prevalence of chronic venous insufficiency in the US has been reported at being up to 40% in adult females and 17% in adult males (Beebe-Dimmer J L, Pfeifer J R, Engle J S, Schottenfeld D, 2005, Ann Epidemiol 15(3):175-84). Some degree of venous insufficiency is considered within the boundaries of normal health. The vast majority of these patients suffer mainly cosmetic changes (varicose veins) or nondebilitating discomfort (mild to moderate swelling of their legs). However, it is estimated that 1,000,000 patients per year in the United States present with chronic distal leg pain with ulcerative or preulcerative changes due to CVI (Ruckley C V, Evans C J, Allan P L, Lee A J, Fowkes F G R, 2002, J Vasc Surg 36:520-525). This most severe group of CVI patients represents the initial focus for the intended device.

As illustrated in FIG. 1, native veins, such as vein 10 with leaflets 11, dilate with increased venous pressure associated with conditions such as chronic venous insufficiency (CVI) and deep vein thrombosis (DVT). As the native veins dilate, fluid velocity 12 decreases and can lead to flow stasis 14 and thrombus formation 16 in the proximity of the valve, further exasperating these disease processes leading to complications including ulcerations. Once a vein segment containing a venous valve has been rendered incompetent, the vein segment may only be repaired if proper flow has been re-established using competent valves. In this case, the vein segment can return to its normal function and dimension. (Meissner et al., 1994, Thrombosis and Haemostasis 72:372-376; Hertzberg et al., 1997, American Journal of Roentgenology 168:1253-1257; and Hertzberg et al., 1998, Journal of Clinical Ultrasound 26:113-117).

Presently, there is no definitive therapy for severe CVI patients. Available treatments are palliative and include pressure stockings and periodic elevation of the extremities to reduce swelling. Ligation and sclerotherapy of veins is used to decrease swelling by forcing increased blood flow from the superficial and perforator veins to the deep veins (Alguire P C, Mathes B M, 1997, J Gen Intern Med, 12:374-383). Attempts to reduce the native venous valve's diameter in situ and restore venous mechanics via thermal denaturation intraluminally or adventitially have failed due to pathophysiologic changes within the venous system associated with CVI (Danielsson et. al., 2003, J Endovasc Ther, 10(2):350-355). Micro-surgical approaches have been difficult to replicate due to demanding techniques and the disease process's effect on the native valve. Previous surgical approaches (direct as well as percutaneous) using synthetic, allograft and/or xenograft prostheses have failed due to toxicity of the implants, thrombosis, and intimal hyperplasia (Neglen P, Raju S, 2003, J Vasc Surg, 37(3):552-557, de Borst G J, et. al., 2003, J Endovasc Ther, 10(2):341-349).

Conventional methods using prostheses for treating CVI in patients have involved the use of stented valvular prostheses placed intraluminally in the vicinity or across a defective native venous valve. However, these stented prostheses either produce non-physiologic flow conditions leading to thrombus and valve failure or cause dilation of the vessels to which they are attached decreasing blood flow rates through the vessels leading to thrombus and valve failure. Examples of stent venous prostheses are described, for example, in U.S. Pat. Nos. 6,287,334, 6,319,281, and 6,503,272, and in U.S. Patent Applications Publication Nos. 20020138135, 20030208261, 20040215339, 20040193253, and 20040260389.

In light of these limitations, there is a pressing need for a device that can restore normal venous circulation to these patients.

### SUMMARY OF THE INVENTION

The invention provides venous valve prostheses comprising: (a) at least one integrally formed venous valve having at least one valve leaflet; (b) a converging nozzle proximal to the valve -and/or a diverging nozzle distal to the valve. FIG. 2 depicts a converging/diverging configuration with the inflow 18 containing the converging section 24 and the outflow 22 containing the diverging section 26 with the valve 20 placed in the middle. FIG. 3 depicts an alternative geometry where the inflow converging nozzle 28 is proximal to the venous valve 32 leading to a continuous diameter outflow 30 smaller than the diameter at the inflow 31. FIGS. 4a and 4b depict venous valve prostheses configurations with multiple valves 36, 37, 42, 44 between the inflow 34, 40 and outflow 38, 46 nozzles.

In certain aspects, a venous valve prosthesis of the invention is derived from a harvested vein segment or is fabricated from a synthetic material. Where the venous valve prosthesis is derived from a harvested vein, the vein can be an allograft or xenograft with respect to the donor and recipient (i.e. the donor of the vein can be a different or the same species as the intended recipient of the venous valve prosthesis of the invention).

In other aspects, a vein containing a venous valve that is harvested as the conduit for a venous valve prosthesis of the invention, is chemically fixed and is manipulated during fixation to create a converging inlet nozzle 24, 28, 34, 40 with a diverging outlet nozzle 26, 38 or a constant diameter length of conduit as a distal nozzle 30, 46. The converging/diverging sections may be of different diametric and length ratios. The valve 20, 32, 36, 37, 42, 44 is positioned such that a converging nozzle 24, 28, 34, 40 is proximal to the valve and a diverging nozzle 26, 38 or constant diameter section of conduit 30, 46 is distal to the valve.

Specific preferred embodiments of the invention will become evident from the following more detailed description of certain preferred embodiments and the claims.

### DESCRIPTION OF THE DRAWINGS

FIG. 1 is a schematic representation of flow stagnation that can lead to thrombus and pannus formation.

FIG. 2 is a representation of a venous valve prosthesis of the invention having both a converging and diverging nozzle.

FIG. 3 is a representation of a converging nozzle with a continuous diameter outflow nozzle.

FIG. 4A is a representation of a venous valve prosthesis of the invention having both a converging and diverging nozzle and multiple valves.

FIG. 4B is an illustration of a converging nozzle with a continuous diameter outflow nozzle and multiple valves.

FIG. 5A, 5B, 5C, and 5D show diagrams of an apparatus for generating a venous valve prosthesis of the invention.

FIG. 6A, 6B are illustrations of a venous valve prosthesis of the invention with proximal and distal ends cut orthogonal to the axis of a graft and obliquely to the axis of the graft.

FIG. 7 is an illustration of a venous valve prosthesis of the invention with proximal and distal ends rolled back to form a cuff.

FIG. 8 is a schematic illustration of the effects of undersized ends of a venous valve prosthesis of the invention at implant (A) and after vein remodeling (B).

FIG. 9A is an illustration of a venous valve prosthesis of the invention implanted in a knee, working with the pumping mechanism of the calf muscle.

FIG. 9B is an illustration of a bicuspid valve oriented such that the line of coaptation of the leaflets is parallel to the bend of the knee.

FIG. 10 is an illustration of a venous valve prosthesis of the invention having second vein segments fixed over the nozzles.

FIG. 11 is an illustration of second vein segments held in place with sutures over the venous valve prosthesis nozzles.

FIG. 12A is an illustration of an isometric view of a inlet converging nozzle.

FIG. 12B is an illustration of a sectioned view (Z-Z′) of an inlet converging nozzle with a linear decrement between the inlet diameter and the outlet diameter of the nozzle.

FIG. 12C is an illustration showing the insertion of an inlet converging fixation nozzle (Sectioned view Z-Z′) into a vein the proximal to the venous valve with a linear decrement between the inlet nozzle diameter and the outlet diameter of the nozzle.

FIG. 13 is an illustration of a sectioned view of an inlet nozzle with a non-linear curvature between the inlet diameter and the outlet diameter of the nozzle.

FIG. 14A is an illustration of placement of a fixtured VVP into the fixation tank.

FIG. 14B is an illustration of the inlet coupling of the fixtured VVP into the fixation tank (Sectioned view X-X′).

### DESCRIPTION OF THE PREFERRED EMBODIMENTS

The invention provides venous valve prostheses, methods of making venous valve prostheses, and methods of use of venous valve prostheses. A venous valve prosthesis of the invention can be used to restore proper venous circulation in a patient by implanting (i.e. grafting) the venous valve prosthesis at a desired location in the patient. For example, implanting a venous valve prosthesis of the invention can be accomplished by surgically suturing the prosthesis to a patient's vein.

A venous valve prosthesis of the invention can be used to bypass a defective venous valve or replace a defective venous valve in a patient in need thereof. Thus, a venous valve prosthesis of the invention can be used to treat a variety of diseases and conditions associated with improper blood circulation, including Chronic Venous Insufficiency (CVI), Deep Vein Thrombosis (DVT), varicose veins, and ulcerations of the extremities.

As used herein, the term “patient” includes human and animal subjects.

As used herein, “treatment” or “treat” refers to both therapeutic treatment and prophylactic or preventative measures. Those in need of treatment include those already having a disorder as well as those prone to have a disorder or those in which a disorder is to be prevented, wherein the disorder is a disease or condition that can be treated by a prosthesis of the invention, such as those described herein.

In certain embodiments, a patient may need multiple venous valve prostheses implanted at various locations. Generally, a venous valve prosthesis of the invention can be implanted as an inter-positional graft using end-to-end, end-to-side, or side-to-side anastomotic techniques within the deep venous system. In some instances, two or more venous valve prostheses may be implanted on the patient's right or left side to restore proper venous flow, one implanted immediately superior or inferior to the knee within the popliteal, common femoral, or superficial femoral veins posterior to the knee and the other implanted along the iliac vein (located in the pelvis). A patient may also require two or more implants on each side (i.e. both the right and left side) to restore proper venous flow. Once proper venous circulation is restored, the reduced peripheral venous pressure will decrease pressure in both the perforating and superficial venous systems making flow in these two systems less problematic.

A venous valve prosthesis of the invention can be derived from a harvested vein segment that contains one or more venous valves (FIGS. 4a-b). Alternatively, a venous valve prosthesis of the invention can be generated from synthetic material including but not limited to urethanes, polyurethane, PTFE, ePTFE, silicones, and other biocompatible polymers known to those skilled in the art. The source of a harvested vein segment can be from a donor of the same species (i.e. an allograft) or from a donor of a different species (i.e. a xenograft). Allograft vein segments are harvested from the peripheral vascular system. In one embodiment, xenograft vein segments can be from jugular veins from equines, bovines, caprines, and ovines. Harvested vein segments to be used to generate a venous valve prosthesis of the invention can have one or multiple valves contained within a single conduit. A segment that comprises multiple valves can be subdivided and used to prepare multiple venous valve prostheses of the invention.

After harvesting, extraneous material, such as muscle, fat, and any other undesired tissue, is preferably removed from the vein. On either side of the valve, an amount of segment remains extended to a length that depends on the particular application and location for the prosthesis. The desired lengths range from about 5.0 mm to about 5.0 cm proximal and about 5.0 mm to about 5.0 cm distal to the segment containing the valve. In certain embodiments, the desired lengths range from about 1.0 cm to about 4.0 cm proximal and about 1.0 cm to about 4.0 cm distal to the segment containing the valve. The segments proximal and distal to the portion of the venous prosthesis containing the venous valve do not have to be of equal length. The harvested vein segment can be manipulated as described herein to a configuration that contains a converging nozzle 24, 28, 34, 40 proximal to the venous valve and a diverging nozzle 26, 38 or constant diameter section of conduit 30, 46 distal to the venous valve.

After harvesting, the xenograft or allograft vein segment is preferably and chemically treated (i.e. cross-linked by chemical fixation as described herein) to render the harvested tissue non-toxic, non-antigenic, and resistant to enzymatic digestion, thereby making the vein segment biocompatible for a desired recipient. In addition, the vein segment is preferably sterilized. One of skill in the art will recognize that methods of sterilization are well known in the art. Biocompatibility is desirable to avoid or minimize fibrous, thrombus, and/-or pannus formation on the venous valve's leaflets in response to recipient tissue and blood, which could cause the leaflets to malfunction leading to prosthesis failure. Several methods of chemically treating xenograft tissues to create biocompatibility are known to those of skill in the art, including, but not limited to chemical cross-linking of the tissue with glutaraldehyde followed by urisal detoxification, 1-ethyl-3-(3-dimethyl aminopropyl) carbodiimide (EDC), and polyglycidyl ether (polyepoxy) compound, as described for example in U.S. Pat. No. 6,166,184, U.S. Pat. No. 6,117,979, and U.S. Pat. No. 5,447,536 each of which is incorporated herein by reference in its entirety. In certain embodiments, chemical treatment can be followed by attaching non-thrombogenic molecules, such as heparin and/or its derivatives, to the vein segment surfaces, which can further reduce potential graft thrombogenicity.

Although allograft venous valve segments do not present the equivalent risk of antigenicity as compared to xenograft venous valve segments, it would still be preferred to treat them with chemical fixation to mitigate bioburden associated with their harvesting and manufacture as well as mitigation of any potential antigenicity. The latter will allow the implant to be used without regard for major histocompatibility matching with the recipient. Likewise, the chemical crosslinking will facilitate maintenance of the converging and diverging nozzle configuration after implantation.

Chemical fixation can be performed so that the valve or valves that are integrally formed in the vein segment will open under forward blood flow conditions and close under backflow pressure. Chemical fixation is preferably performed while the valve or valves that are integrally formed in the vein segment are in an open position (i.e. allowing forward fluid flow), causing the valve to retain an open position after fixation when no back pressure is applied to the valve. The chemical fixation may also occur however with the venous valve leaflets in a semi-open or flaccid position or in a closed position, or with the venous valve leaflets in motion.

Preferably, chemical fixation is performed so that the valve or valves in the vein segment remain open during normal forward blood flow, and are supple enough to close under backflow conditions (see, for example, U.S. Pat. No. 5,500,014, which is incorporated by reference). Generally, a valve of a venous valve prosthesis of the invention will permit flow of blood at a rate of about 0.25 L/min to about 5 L/min, and will close under backflow pressures of less than about 10 mmHg.

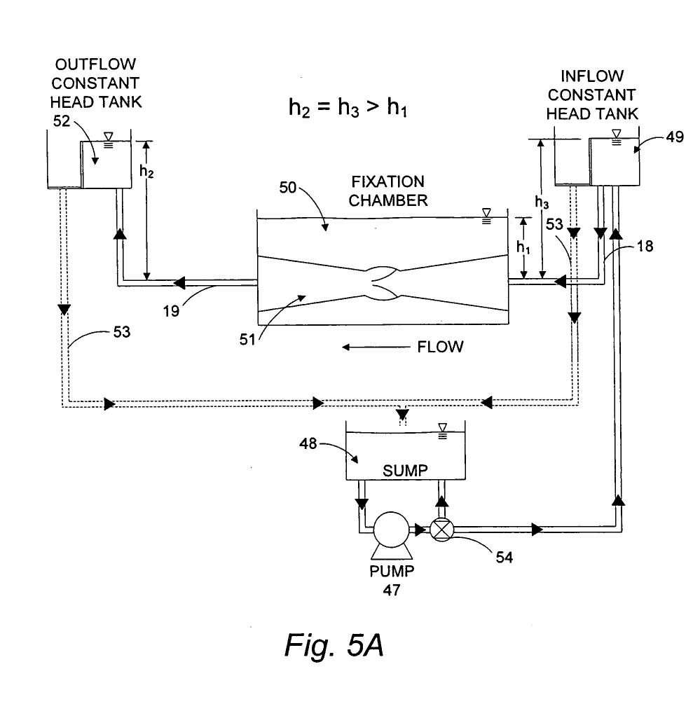

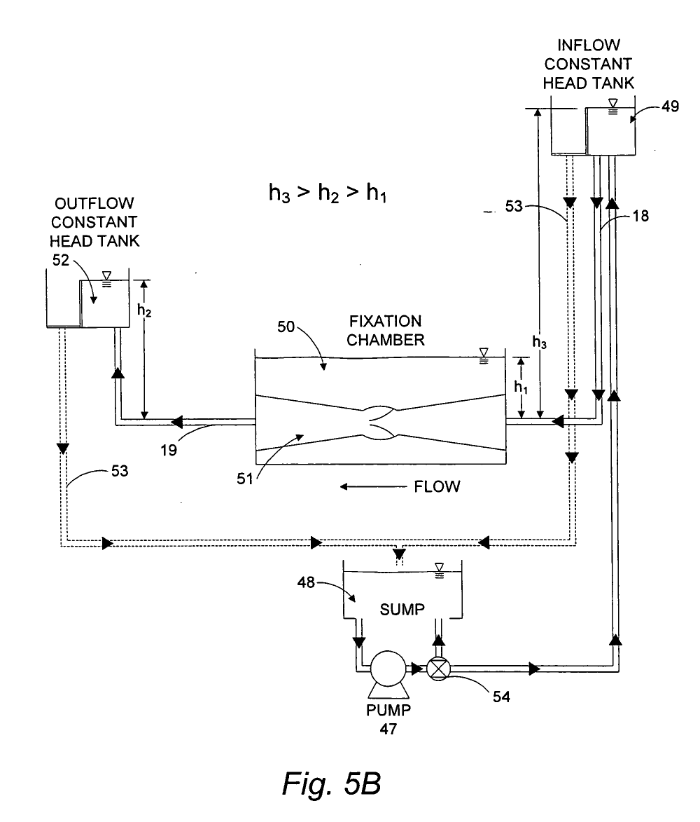

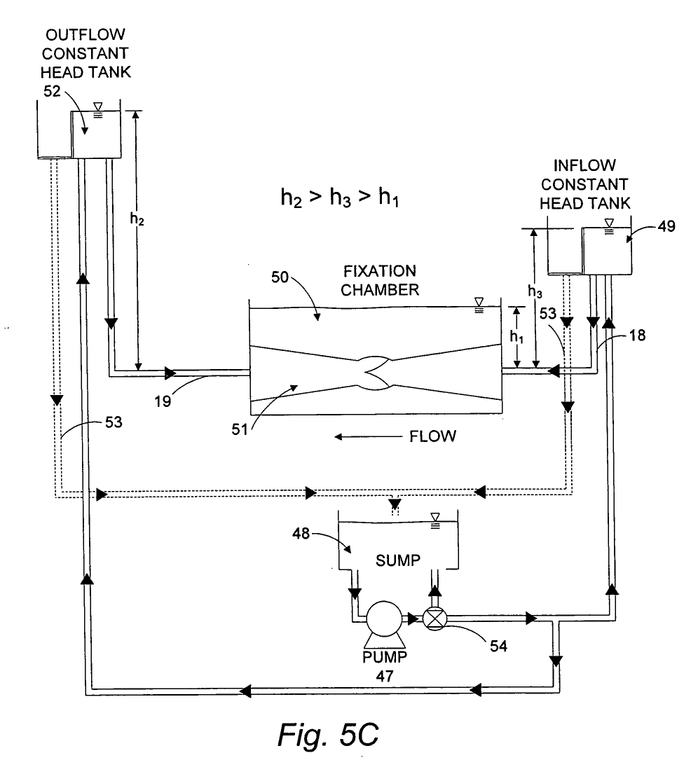

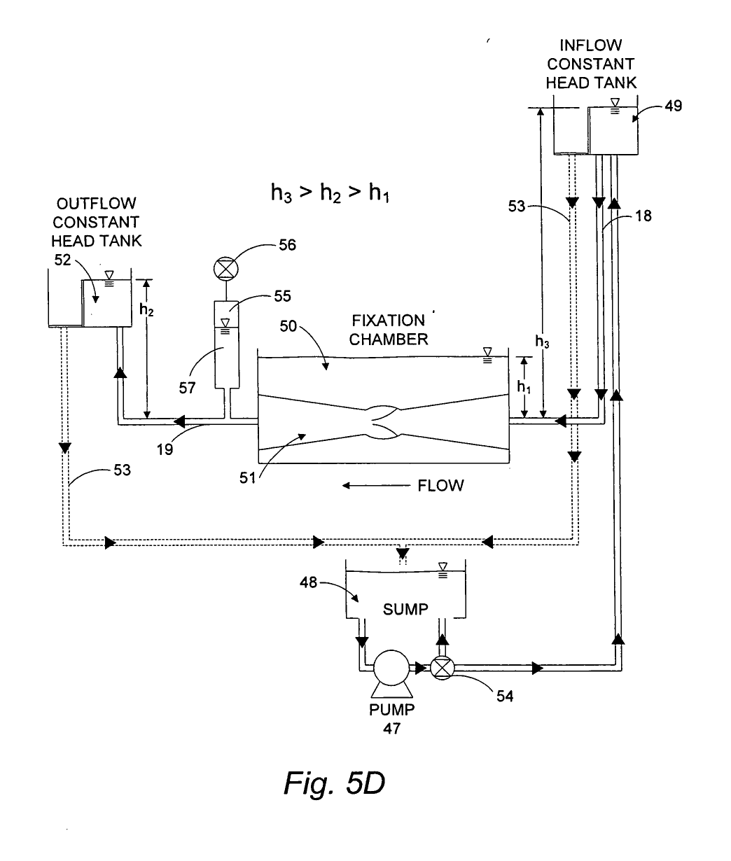

In certain embodiments, the chemical cross-linking can be conducted with the use of a system that supports the valve during the fixation process such that the lumen and adventia of the vein segment is bathed in fixative. FIGS. 5A, 5B, 5C and 5D depict an apparatus for fixation of a xenograft or allograft VVP. The system is designed to allow fixative to bathe the lumen and advential surfaces of the vein containing the venous valve segment. The system shown in FIGS. 5A, 5B, 5C, and 5D represents one design of a hydraulic circuit to accomplish the stated goal of chemical cross-linking of the venous valve prosthesis. In conjunction with the desired fixation chemistry, the system described is designed to allow adjustment of pressure and flow conditions during the fixation process. It is understood that other hydraulic circuit designs exist to those skilled in the art to accomplish the same goal. The pump 47 provides the means to recirculate fluid within the hydraulic circuit. Fluid is provided to the pump 47 via a sump 48. The sump also acts as a reservoir for excess fluid in the hydraulic circuit. The flow rate to the hydraulic circuit is metered by a valve 54 distal to the pump 47. The Inflow Constant Head Tank 49 and Outflow Constant Head Tank 52 are designed to provide a constant flow rate and pressure to the venous valve prosthesis during the chemical fixation process. They each contain a weir to maintain this fluid level. Excess fluid from each constant head tank returns to the sump 48 by a drain tube 53. A Fixation Chamber 50 containing the venous valve prosthesis 51 is placed between the Inflow Constant Head Tank 49 and Outflow Constant Head Tank 52. The venous valve prosthesis 51 is bathed on its advential surface by chemical fixative within the Fixation Chamber 50 and by fluid passing through the hydraulic circuit on its luminal surface. FIG. 5D shows a similar fixation system as FIGS. 5A-5C; however, a pressure chamber 53 has been added between the Fixation Chamber 50 and Outflow Constant Head Tank to impart a pulsatile pressure into the system to force the venous valve leaflets to move in a pulsatile fashion. In this situation, filtered pressurize air 55 is regulated by a valve 56 which alternates between atmospheric pressure and the desired pressure setting. When the valve is open to the pressurized air, it forces the fluid 57 in the pressure tank downward into the flow system superimposing a pressure pulse which closes the venous valve leaflets while the pressure is applied. When the valve 56 is open to atmospheric air, the pressure chamber fluid level 57 returns to its previous height and the venous valve leaflets open. While only one segment is depicted, it is understood that multiple vein segments may be placed into the chamber and connected to the circulating fluid.

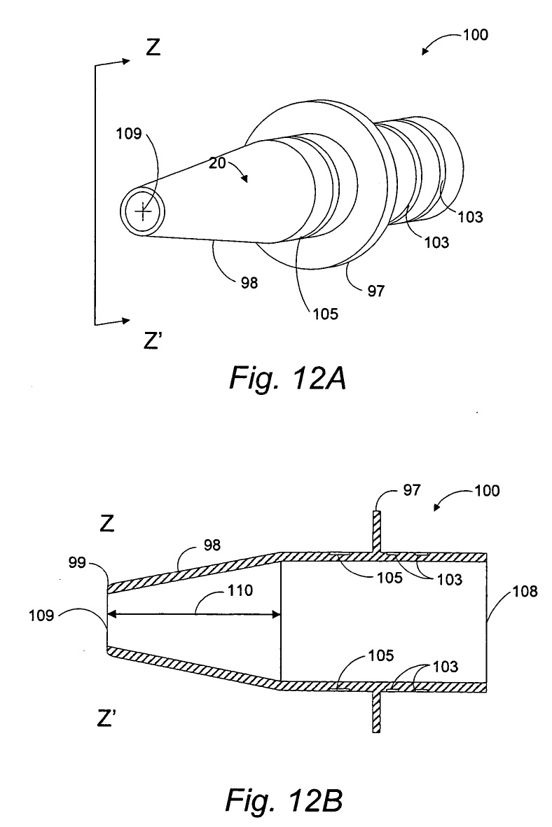

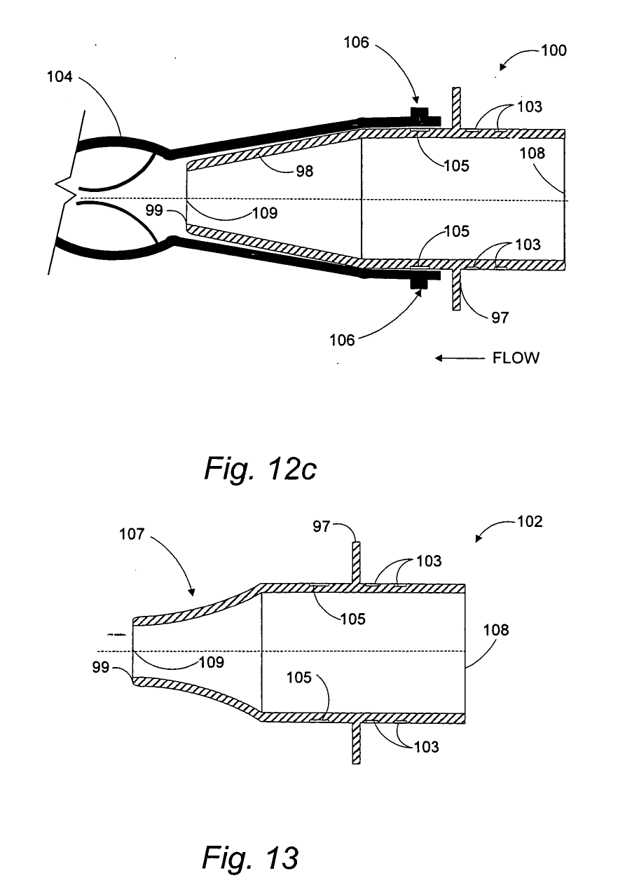

After the tissue vein segment containing the valve is harvested from either allograft or xenograft sources, loose advential tissue is removed and the valve segment is inspected for venous valve structure, competency, and size. An appropriately sized inflow converging fixation nozzle is inserted into the segment of the vein proximal to the venous valve and advanced immediately proximal to the venous valve. FIGS. 12A, 12B, and 13 depict inflow nozzles 100, 102. The nozzles of FIGS. 12A, 12B, and 13 have grooves 103 cut into them to accept o-rings which are used to attach the nozzle to the fixation tank 50. The nozzle flange 97 is used to contact the inner wall surface of the fixation tank wall 109 so that a set distance is maintained between the two nozzles of the fixtured VVP. Another fixation nozzle in a reversed orientation may be used for the distal segment of the vein to create a diverging nozzle configuration for the VVP. The description and drawings provided depict one method of attaching the nozzle to the fixation tank to form fluid tight connections in the hydraulic circuit. It is understood that other means exist to those skilled in the art to form such seals including but not limited to use of gaskets. The outflow nozzle is likewise inserted in the vein segment distal to the venous valve and positioned immediately distal to the venous valve. Care is taken during the insertion process not to damage the tissue or the venous valve. The rounded nozzle configuration 99 minimizes the potential for tissue damage as the nozzle is advanced within the lumen of the vein segment proximal and distal to the venous valve. It is understood that nozzles of other configurations may likewise be inserted.

FIG. 12C depicts the placement of an inflow converging fixation nozzle 100 into the vein segment 104 proximal to the vein segment's venous valve. A seal 106 is made between the nozzle and the tissue, for example by using a tie wrap or O-ring placed over the advential (i.e. outer) surface of the vein at the inflow and outflow, respectively which seat in a groove 105. This serves to compress the venous tissue against the nozzle forming a seal. Other means to form a seal may also be used such as but not limited to glue, clamping, or other compressive methods known to those skilled in the art.

FIGS. 12 A-C depict a fixation nozzle 100 with a linear decrement 98 between the nozzle's inflow diameter 108 and nozzle outflow diameter 109. FIG. 13 depicts a fixation nozzle 102 with a non-linear decrement 107 between the nozzles inflow diameter 108 and outflow diameter 109. It is also understood that the nozzles may be advanced to various positions relative to the venous valve.

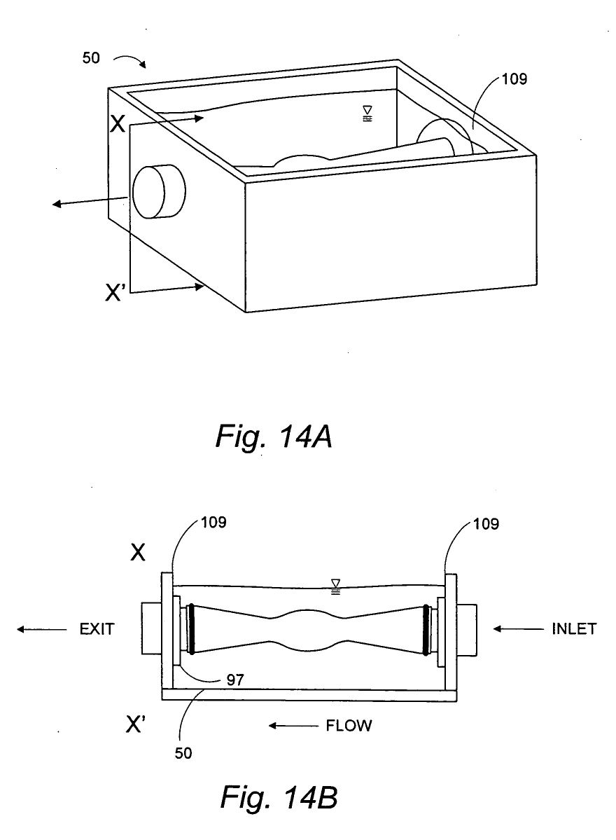

The fixtured venous segment(s) are then placed into the fixation chamber (FIG. 14A and 14B). The fixation chamber 50 is then connected to the fixation system (FIGS. 5A, 5B, 5C and 5D) via hydraulic circuit lines 18 and 19. The opposing ends of the nozzle 108 are of a configuration to allow connection to the fixation system by a mechanical seal. This seal may take the form of an O-ring or other mechanical interlock that would provide a means to separate the lumen of the venous valve from the advential surface when fluid pressure is applied to one or both during the chemical fixation process. Once the hydraulic circuit is connected, it is filled with chemical fixative by pouring fluid into the sump 48 and fixation chamber (FIGS. 14A and 14B). The pump is turned on to remove air bubbles and the outflow valve 54 from the pump is adjusted to provide the desired flow rate leading to the Inflow Constant Head Tank 49. During the priming process, h₃>h₂>h₁ (h=height as measured against the center line of the prosthesis) to remove any air trapped in the lumen of the venous valve. Likewise, the venous segment may be palpated to squeeze any trapped air. Once the entire system is primed, the desired fixation settings may be adjusted. While one VVP is shown in FIGS. 5A, 5B, 5C, and 5D and 14A-B, it is understood that the chamber shown in FIGS. 5A, 5B, 5C, and 5D and 14A-B can contain multiple VVP during the fixation process.

After all air is removed from the system and the VVP is desired to be cross-linked with a chemical fixative under static pressure conditions, the fixation system is configured as shown in FIG. 5A. The lumen is held open via static pressure (0 mmHg<P_{static}<60 mmHg). There is no flow within the lumen because h₂=h₃. In this case, P_{static}=ρg(h₁−h₃) where ρ is the density of the fixation fluid and g is the gravitational constant.

After all air is removed and the VVP is desired to be cross-linked under steady flow (0 L/min<Q<2 L/min, where Q is flow rate) in combination with static pressure (0 mmHg<P_{static}<60 mmHg, where P_{static}=ρg(h₁−h₃)), the system is configured as noted in FIG. 5B with h₃>h₂>−h₁ such that the desired pressure and flow settings are achieved.

After all air is removed and the VVP is desired to be cross-linked with the leaflets closed under back pressure, the system is configured as noted in FIG. 5C with h₂>h₃>h₁ such that the desired pressure settings are achieved.

After all the air is removed and the VVP is desired to be cross-linked under pulsatile flow conditions (0 L/min<Q<2.0 L/min), a pulsatile pressure (1 mmHg<Pₚᵤₗₛₑ<20 mmHg) is superimposed upon the static pressure (0 mmHg<P_{static}<60 mmHg, where P_{static}=ρg(h₁−h₃)) through use of a Windkessel pressure system distal to the vein segment. The solenoid valve 56 of the Windkessel chamber works creates a pressure pulse against the direction of flow. The effect is to create a pulsatile pressure which opens and closes the venous valve. FIG. 5D shows such a configuration. It is also understood that other means exist to those skilled in the art to impart a pulsatile flow to the system. FIG. 5D is intended to depict one option.

Alternatively and using the same systems as noted in FIGS. 5A, 5B, 5C, and 5D, the fixation process can be stopped within the lumen of the VVP by changing the solution in the Sump 48 to a neutral buffer such as saline or phosphate buffered saline while allowing the fixation process to continue in the Fixation Chamber 50 from the advential surface inward toward the lumen of the venous valve prosthesis 51. The advantage of such a system would be that the amount of cross-linking can be controlled to optimize valvular leaflet biomechanics and luminal compliance while rendering the tissue non-antigenic and resistant to enzymatic digestion.

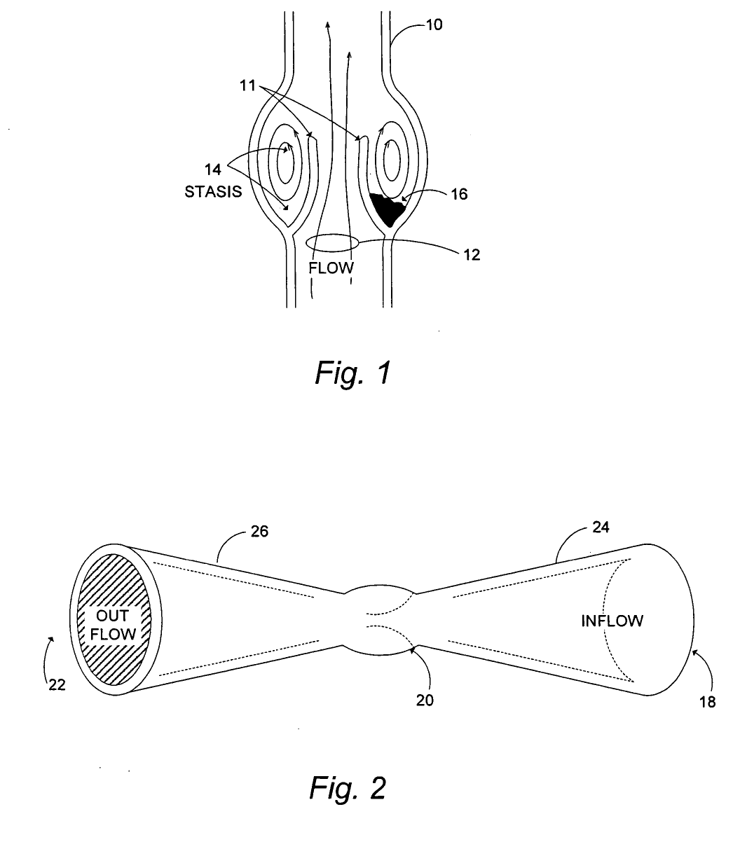

While chemical cross-linking of the tissue removes antigenicity, it also increases the stiffness of both the lumen and valvular leaflet tissue in the harvested vein segment. Fundamentally, the venous valve leaflets become too stiff to open fully under venous pressure and flow conditions. As a result, areas of flow stagnation occur along the distal surfaces of the leaflets and along their insertion into the vein. These areas of flow stagnation can lead to thrombus and pannus formation (FIG. 1). Both result in further restriction of motion and degeneration of the leaflets rendering the valve incompetent and/or stenotic. As discussed below, the configuration of a venous valve prosthesis of the invention overcomes the inherent stiffness of cross-linked leaflets by imparting increased momentum and force to the blood as it enters the converging nozzle and passes through the venous valve leaflets. The increased kinetic energy is converted back to potential energy as the blood moves through the diverging nozzle distal to the venous valve leaflets.

In a particular embodiment of the invention, a vein segment is geometrically manipulated to have the inflow segment proximal to the valve shaped into a converging nozzle using a nozzle form during the chemical fixation process. In another embodiment, the segment distal to the valve is shaped into a diverging nozzle. A venous valve prosthesis having both a converging and diverging nozzle is illustrated in FIG. 2. The converging and diverging nozzles can contain a linear (i.e. consistent) slope or one that is non-linear (e.g. curved, for example, in FIG. 13). The particular configuration will depend, for example, on the desired location of eventual implantation, including the size of the vein to which the venous valve prosthesis will be grafted. In addition, the configuration will depend on the nature of a disease process to be treated (e.g. the severity of the disease), native hemodynamics, and surgical implantation techniques.

The nozzle forms (also referred to herein as “fixation nozzles”) used to shape the inflow and outflow segments can be solid or porous (e.g. an open porous scaffold available from Degradable Solutions AG, Switzerland; a non-absorbable polyvinylidene fluoride (PVDF) mesh, as described in Jansen et al., 2004, Eur. Surg. Res. 36:104-11) so as to allow fixative to pass through the nozzle form through passive diffusion of the chemicals in concert with the pressure gradient between the lumen and advential surfaces of the vein. Prior to the fixation process, the converging nozzle form is inserted into the lumen of the vein segment proximal to the venous valve and the diverging nozzle form is inserted into the lumen of the vein segment distal to the venous valve. These nozzles may be fabricated from materials such as but not limited to metals (such as stainless steel and nitinol), polymers (such as Delrin® (DuPont, Wilmington, Del.) and polycarbonate), metallic screens, or polymeric screens (such as surgical mesh). The end of the nozzle nearest to the venous valve 99 should be of a configuration so as to minimize any potential damage to the tissue during their insertion or during the fixation process (FIG. 12B). The preferred configuration would be to round the edges of the nozzles. The nozzles may also be of a solid or porous configuration. The porosity of the nozzle would act as one means to control the amount of chemical fixative applied to the tissue by adjusting the chemical fixation's ability to diffuse into the tissue as a function of time and concentration.

The nozzle forms also provide the means for precisely controlling the size of the inflow and outflow in a venous valve prosthesis of the invention, which allows appropriate sizing for a particular patient's anatomy (i.e. the size of the vessel to which the prosthesis will be grafted). The nozzle configuration also provides the benefit of increasing product yield by allowing exact sizing of the inlet and exit while a range of sizes can be present in the valve itself. Increasing product yield is important because native tissue (xenograft and allograft) exists in a variety of sizes which would not necessarily match those of the recipient. By forcing the inflow and outflow of the VVP into predetermined sizes, tissue which would have been discarded due to size miss-match with the patient's anatomy would become viable.

Once chemically fixed (i.e. cross-linked), the inflow and outflow sections of the valved venous conduit will maintain their shape. In essence, a Venturi nozzle is created (White F, Fluid Mechanics, 1979 McGraw-Hill Book Company, 166-167), with a venous valve located at the narrowest point. This converging/diverging nozzle configuration serves to accelerate blood flow through the leaflets. The associated increase in blood velocity creates increased force to overcome the increased stiffness in the leaflets associated with the chemical fixation. The ratio of the fixation conduit's largest diameter to its narrowest diameter immediately proximal to the valve may vary. The narrowest conduit diameter of the nozzle can be between about 30% to about 90% of the largest diameter, with the axial length of the fixation nozzle varying between about 5.0 mm to about 5.0 cm. The axial length of the fixation nozzle is illustrated in FIG. 12B as the distance represented by line 110. In certain embodiments, the narrowest conduit diameter of the nozzle can be between about 40%, 50%, 60%, 70%, or 80% of the largest diameter, with the axial length of the fixation nozzle varying between about 1.0 cm to about 4.0 cm.

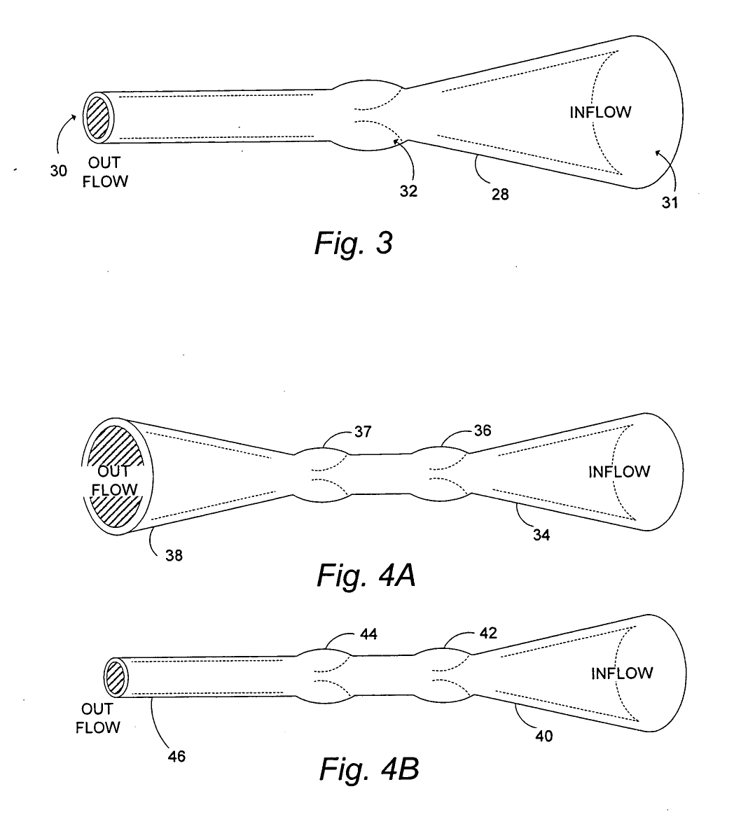

A venous valve prosthesis of the invention can have a variety of configurations. For example, a converging/diverging nozzle configuration of the invention can have a single valve at its narrowest point, as shown in FIG. 2. FIGS. 3 and 4 illustrate additional configurations. FIG. 3 demonstrates a converging nozzle with a continuous diameter outflow nozzle. FIG. 4A-B illustrates concepts shown in FIGS. 2 and 3 with multiple valve segments. The number of valves present in a venous valve prosthesis of the invention will depend on the particular application and needs of the intended recipient.

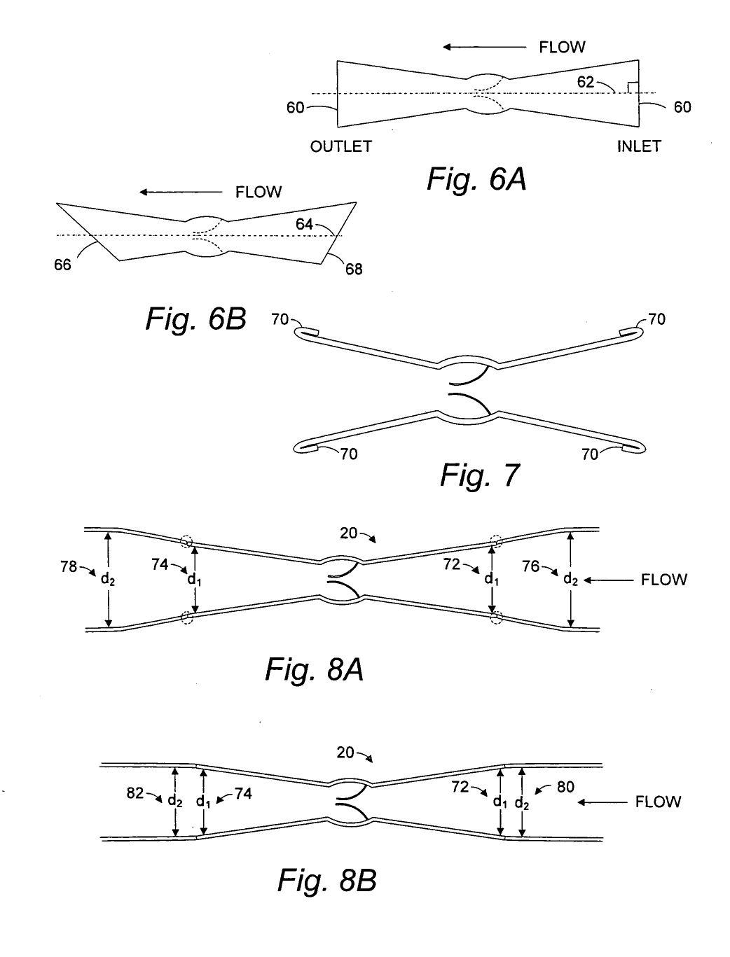

In another embodiment, a venous valve prosthesis can have the proximal and/or distal ends 60 of the venous valve prosthesis cut orthogonal to the long axis of the graft 62 as shown in FIG. 6A. In other embodiments, the proximal 68 and/or distal 66 ends of the venous valve prosthesis can have non-orthogonal or oblique cuts with respect to the axis of the graft 64 as shown in FIG. 6B. It is understood that the proximal and distal ends can both have the same type of cut (e.g. orthogonal and orthogonal) or can have different types of cuts (e.g. orthogonal and non-orthogonal, or orthogonal and oblique). Oblique cuts can be used to create end to end or side to side grafting (i.e. shunting).

In another embodiment, one or both ends of a venous valve prosthesis can be rolled back to form a cuff 70 during the fixation process, as illustrated in FIG. 7. The thicker tissue of the cuff provides that benefit of a durable, pliable surface that resists suture pull out during and after suturing of a venous valve prosthesis of the invention to a recipient vein. A cuff also renders the end of a venous valve prosthesis of the invention amenable to automated suture systems, which hold the host and graft tissue in apposition and place suture, clips, and/or other fastening devices through the tissue to make a seal. A cuff can be created on ends that have been cut orthogonal to the axis of the graft or on ends that have not been cut orthogonal to the graft.

In another particular embodiment, the inflow 72 and/or outflow 74 ends of the venous valve prosthesis 20 of the invention are undersized with respect to the host vein diameters to which it is to be grafted 76, 78 as shown in FIG. 8A. As used herein, “undersized” refers to the smaller diameter of the inflow and/or outflow ends of a venous valve prosthesis compared with the diameter of the recipient host's vein to which the venous valve prosthesis is to be grafted. Under-sizing the ends of the venous valve prosthesis of the invention insures that proper fluid mechanics are preserved even if native tissue remodels as illustrated in FIG. 8B in which the host vein diameters proximal and distal to the VVP remodel such that the interface between the host vein and VVP have diameters at the inflow 72, 80 and outflow 74, 82 approaching each other. The amount of under-sizing will depend on the particular application intended for the venous valve prosthesis of the invention.

Approximately a 20% decrease in diameter is associated with restoration of proper flow subsequent to deep vein thrombosis (DVT) at the popliteal vein (Hertzberg et al. (1997, American Journal of Roentgenology 168:1253-1257). This trend of approximately a 20% reduction in vein diameter over time with proper venous flow restoration applies to areas of the iliac, femoral, and popliteal veins. Thus, in certain embodiments, the inflow and/or outflow end of a venous valve prosthesis of the invention will be undersized about 20% relative to the native vessel to which it is intended to be anastomosed as an interpositional graft. By under-sizing the inflow end of a venous valve prosthesis of the invention relative to the native vessel, blood will naturally accelerate into the venous valve prosthesis.

A venous valve prosthesis of the invention provides several advantages over the conventional valve prostheses such as those discussed herein. Particularly, the two geometric relationships provided herein for a venous valve prosthesis of the invention (converging/diverging nozzle and under-sizing of the graft) is in marked contrast to presently known valve prostheses, which typically use a variety of stented grafts that are deployed percutaneously or placed intraluminally. Such grafts are dilated into place and maintain position via hoop stress against the host vein's luminal surface. In effect, these presently known prostheses are dilating an already dilated segment of graft, which serves to decrease flow velocity and thus reduce the force available to open the leaflets, which creates conditions for flow stasis, thrombus formation, and pannus formation on the leaflets. Combined or singularly, each condition will cause the venous prosthesis to fail. The venous valve prostheses of the invention overcome these shortcomings of the presently known valve prostheses.

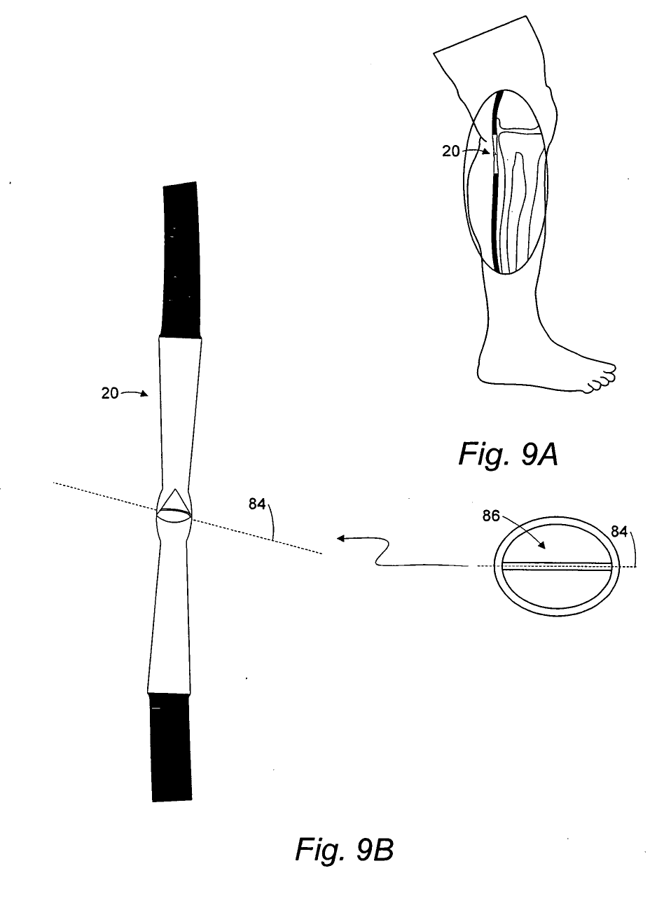

An additional advantage of a venous valve prosthesis of the invention is illustrated in FIGS. 9A and 9B. As shown in FIGS. 9A and 9B, a venous valve prosthesis 20 of the invention can work in concert with the native pumping mechanism due to calf muscle function when walking when it is placed in the popliteal, common femoral, and/or superficial femoral veins (FIG. 9A) as there is no stent to resist the compression of the muscles. This is in marked contrast to stented venous prostheses, which must resist compression so as not to fracture or damage its stent and the valve contained therein. In one embodiment, a venous valve prosthesis of the invention comprises bi-cuspid valve 86 oriented such that the line of coaptation 84 of the leaflets is parallel to the bend of the knee (FIG. 9B), which facilitates maintaining valvular competence even when the knee is bent or the vascular graft is deformed due to muscle contraction. Thus, in a particular embodiment, a venous valve prosthesis of the invention that comprises a bi-cuspid valve that is oriented in the proper plane (i.e. so that the coaptation of the leaflets are parallel to the bend of the knee).

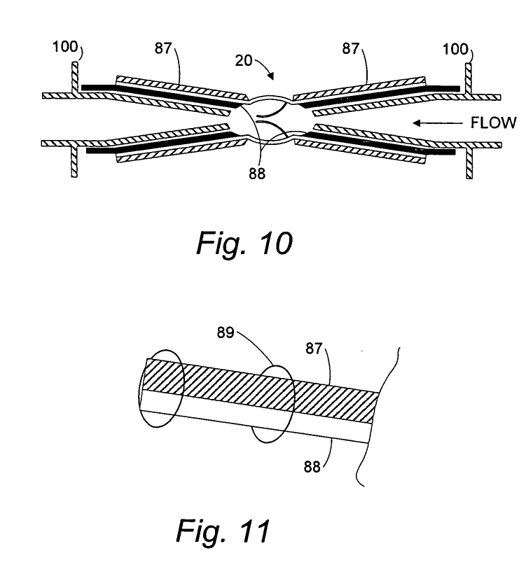

In certain embodiments, an alternative from of a venous valve prosthesis of the invention can be used in which one or more additional vein segments 87, without a valve, are placed over the VVP 20 after fixturing the inflow converging nozzle and/or the outflow diverging nozzle 100 (FIG. 10) during the fixation process. The additional vein segments provide extra durability and strength to the prosthesis and can be used in situations where excessive venous pressure is anticipated. The additional vein segments 87 may be held in place, for example, by sutures 89, adhesives, or physical contact so that there is no relative motion between the lumens of the vein segments 87 and 88 (FIG. 11). The combined vessel thickness will reduce the risk of inlet and outlet conduit dilation insuring valvular competency is maintained.

Alternatively, a venous valve prosthesis of the invention can have a bio-compatible material attached to the outer surface of the venous valve prosthesis. Bio-compatible materials include, but are not limited to, tissues such as allograft or xenograft pericardia, allograft or xenograft fascia, and allograft or xenograft vein segments, and synthetic materials such as urethanes, polyurethane, PTFE, ePTFE, silicones, and other biocompatible polymers known to those skilled in the art.

Unless otherwise required by context, singular terms as used herein shall include pluralities and plural terms as used herein shall include the singular.

It should be understood that the foregoing disclosure emphasizes certain specific embodiments of the invention and that all modifications or alternatives equivalent thereto are within the spirit and scope of the invention as set forth in the appended claims.

What is claimed is:

1. A venous valve prosthesis comprising:
a) at least one integrally formed venous valve having at least one valve leaflet; and
b) a converging nozzle proximal to the valve.

2. The method of claim 1, wherein the converging nozzle has a linear decrement configuration.

3. The method of claim 1, wherein the converging nozzle has a non-linear decrement between the maximum and minimum diameters of the converging nozzle.

4. The venous valve prosthesis of claim 1 further comprising a diverging nozzle distal to the valve.

5. The method of claim 4, wherein the converging nozzle has a linear decrement configuration or a non-linear decrement between the maximum and minimum diameters of the converging nozzle, and wherein the diverging nozzle has a non-linear decrement between the maximum and minimum diameters of the diverging nozzle.

6. The method of claim 4, wherein the converging nozzle has a linear decrement configuration or a non-linear decrement between the maximum and minimum diameters of the converging nozzle, and wherein the diverging nozzle has a linear decrement configuration.

7. The venous valve prosthesis of claim 1, wherein the prosthesis is derived from a harvested vein segment.

8. The venous valve prosthesis of claim 7, wherein the harvested vein segment is an allograft or xenograft.

9. The venous valve prosthesis of claim 7, wherein the prosthesis is chemically treated and sterilized.

10. The venous valve prosthesis of claim 1, wherein one or more vein segments are attached to the outer surface of the venous valve prosthesis.

11. The venous valve prosthesis of claim 1, wherein one or more bio-compatible materials are attached to the outer surface of the venous valve prosthesis.

12. The venous valve prosthesis of claim 1, wherein the prosthesis comprises a synthetic material.

13. The venous valve prosthesis of claim 1, wherein the valve is a one leaflet valve, tri-leaflet valve, or a bi-cuspid valve.

14. The venous valve prosthesis of claim 1, wherein at least one of the distal and proximal ends of the prosthesis is cut orthogonal or oblique to the long axis of the prosthesis.

16. The venous valve prosthesis of claim 1, wherein at least one of the distal and proximal ends is rolled back upon itself.

17. The venous valve prosthesis of claim 1 having an outflow end and an inflow end that are undersized compared with the diameter of a recipient host's vein to which the venous valve prosthesis is to be grafted.

18. A method of facilitating natural pumping mechanism of the calf muscles to reduce venous pressure in a patient in need thereof, the method comprising implanting the venous valve prosthesis of claim 1 into said patient.

19. The method of claim 18, wherein the venous valve prosthesis comprises a bi-cuspid valve oriented so that the coaptation of the leaflets of the valve are parallel to the bend of the patient's knee.

20. A method for making the venous valve prosthesis of claim 1, the method comprising the steps of:
(a) harvesting a vein segment comprising a venous valve;
(b) inserting a converging fixation nozzle into the vein proximal to the venous valve;
(c) optionally inserting a diverging fixation nozzle into the vein distal to the venous valve;
(d) placing the vein segment into a fixation chamber;
(e) removing air bubbles from the vein segment; and
(f) contacting the outer surface and lumen of the vein segment with a chemical fixative.

21. The method of claim 20, wherein either or both of the fixation nozzles are porous.

22. The method of claim 20, wherein either or both of the fixation nozzles are non- porous.

23. The method of claim 20, wherein the chemical fixative contacts the lumen of the vein segment under static, steady forward flow, steady back pressure, or pulsatile conditions.

24. The method of claim 20, wherein either or both of the fixation nozzles have an axial length of about 5.0 mm to about 5.0 cm.

25. The method of claim 20, wherein either or both of the fixation nozzles have a narrowest diameter of about 30% to about 90% of the largest diameter of the venous valve prosthesis.

26. The method of claim 20, wherein either or both of the fixation nozzles have a linear decrement configuration.

27. The method of claim 20, wherein either or both of the fixation nozzles have a non-linear decrement between the maximum and minimum diameters of the fixation nozzles.

28. The method of claim 20, wherein the edges of either or both of the fixation nozzles are rounded.

29. The method of claim 20, further comprising the step of stopping the contact of chemical fixative within the lumen of the vein segment by replacing the chemical fixative in the lumen with a buffer while the chemical fixative remains in contact with the outer surface of the vein segment.
